(19) United States
(12) Patent Application Publication
(10) Pub. No.: US 2011/0306110 A1
(43) Pub. Date: Dec. 15, 2011

(54) METHOD FOR THREE-DIMENSIONAL HIERARCHICAL CELL CO-CULTURE

(75) Inventors: Shoji Takeuchi, Tokyo (JP); Yukiko Matsunaga, Tokyo (JP); Yuya Morimoto, Tokyo (JP)

(73) Assignee: THE UNIVERSITY OF TOKYO, Tokyo (JP)

(21) Appl. No.: 13/133,624

(22) PCT Filed: Dec. 14, 2009
(86) PCT No.: PCT/JP2009/071087

§ 371 Date: Jun. 8, 2011

(30) Foreign Application Priority Data
Dec. 12, 2008 (JP) 2008-317519

(51) Int. Cl. C12N 5/071; C12N 11/10; C12N 5/0775; C12N 11/02

(52) U.S. Cl. 435/178; 435/397; 435/177; 435/347

(57) ABSTRACT

A method for preparing a biomaterial comprising a gel layer which forms a core region, and cells (cover cells) which cover around the gel layer, said method comprising the steps of:

(a) bringing a biocompatible oil or a vegetable oil or a mixture thereof with a mineral oil into contact with a solution containing a gel-forming component to form monodisperse droplets;

(b) inducing gelation of the monodisperse droplets to give gel beads; and

(c) seeding the cover cells over the surface of the gel beads.

### TECHNICAL FIELD

The present invention relates to a method for three-dimensional hierarchical cell co-culture using microgel capsules.

### BACKGROUND ART

To mimic or reproduce tissues and organs in vitro, various types of cell-cell interactions are required.

The inventors of the present invention have found that cell-cell interactions affect the activation of cellular functions in conventional two-dimensional co-culture systems (S. N. Bhatia et al., FASEB J., 13 (1999), pp. 1883-1900; Y. Tsuda et al., Biochem. Biophys. Res. Comm., 348 (2006), pp. 937-944). However, native tissues in the body are structured hierarchically in three dimensions, and it is therefore imperative to co-culture different types of cells in three dimensions rather than in two dimensions (FIG. 1).

Techniques used for three-dimensional culture involves dispersing different types of cells in a gel, or preparing cell sheets and stacking them together.

However, when different types of cells are dispersed in a gel by conventional three-dimensional culture techniques, the cells tend to aggregate and hence it is nearly impossible to control the orientation of each type of cells (A. Ito et al., J. Biosci. Bioeng., 104 (2007), pp. 371-378). Moreover, since cells have a tendency to cause aggregation between those of the same type, it is difficult to achieve three-dimensional culture of different cell types when simply co-culturing different types of cells. For these reasons, hierarchical co-culture has been nearly impossible.

Further, when cell sheets are used for co-culture, the cells can be stacked and arranged hierarchically. However, it has been difficult to construct a three-dimensional hierarchical structure which is reproduced in vivo on a micro scale.

### Non-patent Document 1: S. N. Bhatia et al., FASEB J., 13 (1999), pp. 1883-1900

Non-patent Document 2: Y. Tsuda et al., Biochem. Biophys. Res. Comm., 348 (2006), pp. 937-944
Non-patent Document 3: A. Ito et al., J. Biosci. Bioeng., 104 (2007), pp. 371-378

### DISCLOSURE OF THE INVENTION

For these reasons, there has been a demand for the development of a technique in which different types of cells are arranged within and on a biomaterial, which is biocompatible and serves as a scaffold for the cells, to thereby three-dimensionally reconstruct their native structures and functions.

The present invention aims to provide a method for three-dimensional hierarchical cell co-culture.

As a result of extensive and intensive efforts made to solve the above problems, the inventors of the present invention have succeeded in preparing a three-dimensional hierarchical biomaterial using a device called AFFD by contacting a cell-containing solution and an oil at a given ratio in a chamber that is used to join two flows into one to thereby prepare microdroplets of uniform diameter, encapsulating first cells (serving as the core) within the microdroplets, and then forming a layer of second cells over the outer surface (gel surface) of the microdroplets. This finding led to the completion of the present invention.

Namely, the present invention is as follows.

(1) A method for preparing a biomaterial comprising a gel layer which forms a core region, and cells (cover cells) which cover around the gel layer, said method comprising the steps of:

(a) bringing a biocompatible oil or a vegetable oil or a mixture thereof with a mineral oil into contact with a solution containing a gel-forming component to form monodisperse droplets;

(b) inducing gelation of the monodisperse droplets to give gel beads; and

(c) seeding the cover cells over the surface of the gel beads.

(2) A method for preparing a biomaterial comprising a gel layer which forms a core region where cells (core cells) are encapsulated, and cells (cover cells) which cover around the gel layer, said method comprising the steps of:

(a) bringing a biocompatible oil or a vegetable oil or a mixture thereof with a mineral oil into contact with a solution containing the core cells and a gel-forming component to form monodisperse droplets;

(b) inducing gelation of the monodisperse droplets to give gel beads; and

(c) seeding the cover cells over the surface of the gel beads.

(3) A method for preparing a biomaterial comprising a gel layer which forms a core region or a gel layer which forms a core region where cells (core cells) are encapsulated, cells (cover cells) which cover around the gel layer, and an additional set of a gel layer and cover cells coated sequentially over the first-mentioned cover cells, said method comprising the steps of:

(a) bringing a biocompatible oil or a vegetable oil or a mixture thereof with a mineral oil into contact with a solution containing a gel-forming component or with a solution containing the core cells and a gel-forming component to form monodisperse droplets;

(b) inducing gelation of the monodisperse droplets to give gel beads;

(c) seeding the cover cells over the surface of the gel beads; and

(d) preparing a mixture containing the biomaterial obtained in step (c) and a gel-forming component, and then repeating steps (a) to (c) by using the biomaterial as the core cells in step (a).

In the present invention, examples of a gel-forming component include extracellular matrix components. In addition, a preferred vegetable oil is corn oil, and a preferred mineral oil is liquid paraffin. In this case, the mixing ratio between vegetable oil and mineral oil is, for example, 1:2.

In the present invention, extracellular matrix components may be exemplified by at least one member selected from the group consisting of collagen, proteoglycans, glycosaminoglycans, fibronectin, laminin, tenascin, entactin, elastin, fibrin, hyaluronic acid, gelatin, alginic acid, agarose and chitosan, by way of example.

In the present invention, monodisperse droplets can be formed when the above oil mixture and the above solution meet each other in a device comprising a first channel through which the above oil mixture flows and a second channel through which the above solution flows.

In one embodiment of the present invention, the core cells are derived from liver cancer and the cover cells are fibroblasts.

(4) A method for producing a cell aggregate having hierarchically structured cell layers, which comprises culturing the biomaterial obtained by the above method.

(5) A method for producing a reconstructed tissue, which comprises culturing the biomaterial or cell aggregate obtained by the above method within a mold of any shape.

(6) A method for preparing a hollow biomaterial comprising a gel layer which has a hollow cavity and forms a core region, a biomolecule or cells which are encapsulated within the hollow cavity, and cells (cover cells) which cover around the gel layer, said method comprising the steps of:

(a) bringing a biocompatible oil or a vegetable oil or a mixture thereof with a mineral oil into contact with a solution containing cells or a biomolecule and a gel-forming component to form monodisperse droplets;

(b) inducing gelation of the monodisperse droplets to give gel beads;

(c) coating the surface of the gel beads with another gel component different from said gel and then inducing gelation; and

(d) dissolving the gel obtained in step (b).

(7) The present invention further provides a cell aggregate or tissue obtained by the method according to any one of (1) to (5) above.

(8) The present invention further provides a method for producing a cell aggregate or biomolecular aggregate having a hollow cavity, which comprises culturing the hollow biomaterial obtained by the method according to (6) above. The present invention also provides a cell aggregate or biomolecular aggregate having a hollow cavity obtained by this method.

(9) A hierarchically structured cell aggregate comprising a gel layer which forms a core region, and a layer of cells which cover around the gel layer.

(10) A hierarchically structured cell aggregate comprising a gel layer which forms a core region where cells (core cells) are encapsulated, and a layer of cells (cover cells) which are different from the core cells and cover around the core cell layer.

(11) A hierarchically structured cell aggregate comprising a gel layer which forms a core region, a layer of cells (cover cells) which cover around the gel layer, and one or more cell layers coated sequentially with cells of the same or different type from the cover cells.

(12) A hierarchically structured cell aggregate comprising a gel layer which forms a core region where cells (core cells) are encapsulated, a layer of cells (cover cells) which are different from the core cells and cover around the core cell layer, and one or more cell layers coated sequentially with cells of the same or different type from the cover cells.

In one embodiment of the above cell aggregate of the present invention, the core cells are HepG2 cells and the cover cells are 3T3 cells.

(13) A reconstructed tissue assembled from the cell aggregates according to any one of (7) to (12) above.

(14) An in vivo-like model of liver tissue comprising the above cell aggregate(s) or a reconstructed tissue assembled from said cell aggregates.

(15) The method according to (5) above, wherein the cell density is uniform throughout the tissue.

(16) The tissue according to (7) or (13) above, wherein the cell density is uniform throughout the tissue.

The present invention provides biomaterials having hierarchical cell layers and a method for their preparation. Moreover, the method of the present invention enables the preparation of monodisperse gel droplets encapsulating cells.

In the Example section described later, an oil mixture consisting of corn oil and a mineral oil is used for gelation to prepare monodisperse collagen gel beads, within which the viability of the encapsulated cells can be maintained. A system with these collagen gel beads is useful for analysis and experiment of three-dimensional tissue co-culture. In the Example section, the inventors of the present invention used 3T3 and HepG2 cells as model cells and succeeded in hierarchical three-dimensional co-culture of these cells. The inventors also demonstrated that the presence of 3T3 cells increased the rate of albumin secretion from HepG2 cells. According to this result, liver functions can be reproduced more precisely as compared to the results measured for albumin secretion rate in two-dimensional co-culture under the same conditions, and it is therefore possible to understand the mechanisms of three-dimensional hierarchical cell-cell interactions in organs. Moreover, the monodispersity of collagen beads is an important property required to prepare bead arrays for bead-based microchannel array systems, and allows quantitative analysis of cells for their physiological functions and drug responsibility. Thus, the three-dimensional tissue co-culture technique, which is completed by the method of the present invention, can be regarded as an economical and convenient tool for in vitro study of in vivo-like microenvironments and cell-cell interactions.

### BRIEF DESCRIPTION OF THE DRAWINGS

FIG. 1 shows the concept of three-dimensionally co-cultured microtissues, in comparison with two-dimensional culture. These three-dimensionally cultured tissues provide microenvironments similar to organs and tissues as compared to conventional two-dimensional co-culture.

FIG. 2 shows a schematic diagram of AFFD fabricated by stereolithography to prepare monodisperse collagen droplets, and a process flow for preparing collagen beads by three-dimensional co-culture using two types of cells.

FIG. 3 shows the morphology of microbeads.

Panel (a) shows a monodisperse image of collagen droplets in corn oil, and panel (b) shows a monodisperse image of collagen gel beads in culture medium. In panel (b), collagen gel beads are visualized with subnano-sized beads (red) and cell nuclei are stained with Hoechst 33342 (blue). Panels (c) and (d) show the diameter distribution of the collagen droplets in panel (a) and the collagen beads in panel (b), respectively. It is indicated that the collagen beads retain their monodispersity even after collagen gelation. Panel (e) is a confocal laser scanning microscopic image of a collagen bead encapsulating cells after being incubated for 30 hours. Upon visualization of living cells with a Live/Dead assay kit, most of the cells are alive within the collagen beads (living cells are shown in green). Moreover, the surviving cells are found to grow along the shape (spherical shape) of collagen beads.

FIG. 4 shows the morphology of microbeads.

Panel (a) shows the concept of collagen beads coated with 3T3 cells, and panel (b) is an image of collagen beads coated with 3T3 cells after being cultured for 24 hours. Panels (c) and (d) are images of gel beads coated with 3T3 cells after being cultured for 24 and 30 hours, respectively. 3T3 cells were visualized with a Live/Dead Assay kit (green). The 3T3 cells adhered on the collagen beads were found to grow and cover the surface of the gel beads gradually. Panel (e) is a confocal microscopic image of 3T3 cells after being cultured for 30 hours. 3T3 cells were visualized with a Live/Dead Assay kit (living cells are shown in green and dead cells are shown in red), and cell nuclei were stained with Hoechst 33342 (blue). The 3T3 cells were found to form a layer on the surface of the collagen beads and most of the cells were alive even after 30 hours.

FIG. 5 shows the morphology of microbeads.

Panel (a) shows the concept of HepG2 encapsulated within collagen beads. Panel (b) is a bright field image of HepG2 cells within collagen beads, and panel (c) is an image showing HepG2 cells visualized with a Live/Dead Assay kit (green) and cell nuclei stained with Hoechst 33342 (blue). Most of the HepG2 cells encapsulated within collagen beads were alive during gelation.

FIG. 6 shows the morphology of microbeads.

Panel (a) shows a confocal microscopic image of collagen beads after three-dimensional co-culture for 30 hours. HepG2 cells were stained red, while 3T3 cells were stained green. 3T3 cells are clearly distinguished from HepG2 cells. Panel (b) is an image showing albumin secretion from HepG2 cells. Albumin was stained by immunostaining (green). It was confirmed that HepG2 cells co-cultured with 3T3 cells secreted albumin.

FIG. 7 is a graph showing the immunofluorescence intensity of albumin secretion from HepG2 cells cultured under different culture conditions. This result indicates that HepG2 cells show an increased rate of albumin secretion when confined by 3T3 cells.

FIG. 8 is a process flow for preparing hollow microbeads having a hollow cavity for holding cells or biomolecules.

FIG. 9 is a process flow for forming a reconstructed tissue by assembling the cell aggregates of the present invention.

FIG. 10 is a process flow illustrating how to produce a reconstructed tissue from cells by molding techniques.

FIG. 11 is a process flow illustrating how to produce a reconstructed tissue from a combination of various cell aggregates by molding techniques.

FIG. 12 shows tissues reconstructed from cell aggregates in a human-shaped mold.

FIG. 13 shows time-induced changes in a tissue obtained by the method of the present invention.

FIG. 14 shows the results tested for the viability of cell aggregates in a tissue at 30 hours after the initiation of reconstruction (Live/Dead assay).

FIG. 15 shows confocal microscopic images of 3T3 cell gel beads and 3T3 cell spheroids, as well as H.E. stained images of whole tissues and tissue sections at 24 hours after the initiation of reconstruction.

FIG. 16 shows the results of cell density at different sites determined from tissue sections.

FIG. 17 shows a three-dimensional heterotissue formed from HUVEC cell gel beads and NIH/3T3 cell gel beads by molding.

FIG. 18 shows reconstructed tissues obtained by co-culture using two types of cell aggregates.

### EXPLANATION OF REFERENCE NUMERALS

201: channel, 202: first chamber, 203: channel, 204: second chamber, 205: outlet, 206: first cell, 207: solution containing extracellular matrix components, 208: monodisperse droplet, 209: narrow orifice, 210: channel

800: microbead, 801: first gel component, 802: second gel component, 803: cell or biomolecule, 810: double-layered gel microbead, 820: inner wall, 830: cell, 840: hollow cavity, 880:cell aggregate

### BEST MODES FOR CARRYING OUT THE INVENTION

The present invention will be further described in more detail below. The scope of the present invention is not limited to the following description, and any embodiments other than those illustrated below may also be carried out with appropriate modifications without departing from the spirit of the invention.

It should be noted that all publications cited herein, including prior art documents, patent gazettes and other patent documents, are incorporated herein by reference. This specification incorporates the contents disclosed in the specification of Japanese Patent Application No. 2008-317519 (filed on Dec. 12, 2008), based on which the present application claims priority.

### 1. Overview

The present invention is intended to form uniform micro-sized droplets by using microchannels, with or without encapsulating cells within these droplets. The droplets thus formed are then gelled and transferred to a culture solution, whereby microdroplets (also referred to as “gel beads”) of uniform size can be prepared while keeping the encapsulated cells in a viable condition. When cells of another type are seed on these gel beads, it is possible to obtain a biomaterial in which one or more types of cells are arranged three-dimensionally via the gel beads. When this biomaterial is then cultured in an incubator, the cells will consume collagen and other extracellular matrixes, which are components of the gel, as their nutrient sources. For this reason, if the gel beads encapsulate cells, the cells thus encapsulated (referred to as “core cells”) and the cells seeded outside (over the surface) of the gel beads (referred to as “cover cells”) are contacted with each other to form cell aggregates. In the case of using collagen to prepare gel beads, the thus prepared gel beads are referred to as “collagen gel beads” (collagen gel beads) or “collagen beads.”

Upon cell-to-cell contact as described above, either or both of the encapsulated core cells or the outer-layered cover cells will exert their original functions, so that intercellular interactions can be confirmed indirectly and/or directly.

Thus, the present invention provides a method for preparing a biomaterial comprising a gel layer which forms a core region, and cells (cover cells) which cover around the gel layer, said method comprising the steps of:

(a) bringing a biocompatible oil or a vegetable oil or a mixture thereof with a mineral oil into contact with a solution containing a gel-forming component to form monodisperse droplets;

(b) inducing gelation of the monodisperse droplets to give gel beads; and

(c) seeding the cover cells over the surface of the gel beads.

The present invention also provides a method for preparing a biomaterial comprising a gel layer which forms a core region where cells (core cells) are encapsulated, and cells (cover cells) which cover around the gel layer (i.e., a biomaterial having hierarchically structured cell layers), said method comprising the steps of:

(a) bringing a biocompatible oil or a vegetable oil or a mixture thereof with a mineral oil into contact with a solution containing the core cells and a gel-forming component to form monodisperse droplets;

(b) inducing gelation of the monodisperse droplets to give gel beads; and

(c) seeding the cover cells over the surface of the gel beads.

In the present invention, it is possible to construct three-dimensional hierarchical cell layers similar to those observed in vivo in an experimental environment because cells are co-cultured via gel beads. The biomaterials and cell aggregates of the present invention are characterized in that they float independently in a fluid, unlike cell arrays in which materials or cells are immobilized on an array substrate. This allows observation of interactions between different types of cells in an environment more similar to in vivo conditions than that achieved by conventional methods. Moreover, the gel beads and biomaterials have uniform shape and size, which facilitates quantitative evaluation and easy handling. In recent years, there have been proposed high-throughput devices for screening, by which spherical capsules of uniform diameter are arranged in a given pattern for the purpose of easy analysis. Thus, these devices will have a wider range of applications when combined with the biomaterials or cell aggregates completed by the present invention.

The biomaterials and cell aggregates constructed by the inventors of the present invention have two or more hierarchical cell layers and provide in vivo-like microenvironments (tissue models). In the context of the present invention, the term “hierarchical cell layers” does not refer to layers stacked in sheet form. It is intended to mean layers of two or more types of cells in which one cell population is entirely covered with another cell population and each cell population forms a layer to give a three-dimensional thickness. Thus, the biomaterials of the present invention are not limited in any way as long as they do not have a sheet-like stacked structure, although they are preferably spherical (bead-shaped), by way of example. Such biomaterials in spherical form are also herein referred to as microbeads.

Further, in the present invention, once the above biomaterials or cell aggregates have been assembled, they will be reconstructed and self-organized to form a tissue. This reconstructed tissue allows reproduction of an in vivo-like tissue model much more similar to an actual tissue in the body.

### 2. Preparation of Biomaterials

### (1) Gel Layer Which Forms a Core Region

In the present invention, the gel layer which forms a core region includes two embodiments where it composed of a gel alone and where cells are encapsulated within a gel.

### (1-1) Embodiment Where Core Cells are Encapsulated

Cells which serve as the core to be encapsulated within a gel, i.e., cells which form the innermost region of the biomaterial of the present invention are referred to as “core cells.” Such core cells are not limited in any way and may be appropriately selected from animal cells, plant cells and so on, depending on the intended purpose. They may be cells collected from the body, established cell lines, or even tumor cells. Since the present invention aims to achieve in vitro construction of an environment composed of in vivo-like three-dimensional cell constructs, the core cells are preferably capable of exerting their functions upon interaction with cover cells (described later) which are used to cover the surface of gel beads (the surface layer of the gel). Examples include liver cells, as exemplified by liver parenchymal cells (hepatic cells), as well as a group of cells called liver non-parenchymal cells, including sinusoidal endothelial cells, Kupffer cells, astrocytes, pit cells, biliary epithelial cells and so on. Other examples include pancreatic cells such as α cells and β cells. As shown in the Example section described later, it is also possible to use hepatoma cells (e.g., HepG2 cells). Further, cells called pluripotent cells, i.e., ES cells, iPS cells, and mesenchymal stem cells collected from bone marrow may be co-cultured with feeder cells of another cell type which serve as an aid for adjusting their culture conditions.

Thus, in the present invention, a combination of pluripotent cells and feeder cells (one of which is used as core cells and the other as cover cells described later) can also be listed as an example.

### (1-2) Embodiment Where Core Cells are not Encapsulated

In the present invention, the gel layer which forms a core region also includes an embodiment where cells (core cells) are not encapsulated. For example, if the core cells are not required to exert their functions upon interaction with cover cells, the core cells may not be encapsulated in the present invention and the core region is formed from a gel-forming component alone.

### (2) Cells Which Constitute a Surface Layer Region of the Gel

In the present invention, cells used to cover the outside of the gel, i.e., cells used to cover the gel layer which forms a core region in the biomaterial of the present invention are referred to as “cover cells.” The phrase “cover the gel layer” is intended to mean covering the surface of the gel, but in actual fact, the cover cells infiltrate somewhat into the gel and exist within a layer of the outermost region of the gel to thereby constitute a surface layer region of the gel. These meanings are collectively used herein under the phrase “cover the gel layer.” The cover cells are not limited in any way, but in a case where cells are encapsulated within the core region, the cover cells preferably have the ability to interact with the encapsulated core cells. Thus, the cover cells may be selected as appropriate for the type of core cells to be encapsulated. Examples include fibroblasts in the case of using liver cells as core cells, mouse fetal fibroblasts or the like in the case of using ES cells or iPS cells, as well as α cells in the case of using pancreatic islet β cells. As shown in the Example section described later, fibroblasts such as 3T3 cells are preferred for use when HepG2 cells are used as first cells.

Of course, core cells and cover cells may be interchanged with each other in some embodiments of the present invention.

### (3) Gel-Forming Component

The gel-forming component used in the present invention refers to any material serving as a scaffold for cells, which is selected from:

(i) extracellular matrix components (which may be contained in gelatin);

(ii) naturally occurring materials such as gelatin, chitosan, agarose or the like; and

(iii) synthetic materials such as peptide gels, polyethylene glycol, polylactic acid or the like.

Examples of extracellular matrix include the following materials, which are listed for illustrative purposes only and are not intended for limitation:

collagen, proteoglycans, glycosaminoglycans, fibronectin, laminin, tenascin, entactin, elastin, fibrin, alginate, and agarose.

Examples of proteoglycans include chondroitin sulfate proteoglycan, heparan sulfate proteoglycan, keratan sulfate proteoglycan, dermatan sulfate proteoglycan, etc.

As a member of glycosaminoglycans, hyaluronic acid can be presented.

Examples of synthetic materials include supramolecular peptide gels having specific amino acid sequences (PuraMatrix, Panacea gel), as well as synthetic polymers such as polyethylene glycol, polylactic acid, polyglycolic acid, etc.

These components may be selected alone or in combination, as appropriate, or may be modified to encapsulate cell adhesion factor and growth factor before use.

### (4) Oil or Oil Mixture

In the present invention, a biocompatible oil or a vegetable oil or a mixture thereof with a mineral oil is used. The oil or oil mixture used in the present invention refers to any solution immiscible with water, and examples of such an oil mixture include combinations of a biocompatible oil and a mineral oil, combinations of a vegetable oil and a mineral oil, or combinations of a biocompatible oil, a vegetable oil and a mineral oil, etc. The oil or oil mixture is used during gelation of the gel-forming component. The oil or oil mixture may be supplemented as appropriate with additives, if necessary.

For convenience of explanation, the biocompatible oil used in the present invention is distinguished herein from the vegetable oil described below. However, it does not mean that the vegetable oil used in the present invention is not biocompatible. It is of course possible to use a biocompatible vegetable oil, and it is also possible to use even a vegetable oil not necessarily compatible in the body as long as it is suitable for preparing the biomaterials of the present invention.

Examples of such a biocompatible oil include fluorocarbons. The term “fluorocarbons” is used as a generic name for organic compounds having carbon-fluorine bonds. Since fluorocarbons are less likely to cause chemical reactions and are stable against changes in temperature, they can also be used in the present invention. Hydrocarbons whose hydrogen atoms are all replaced with fluorine atoms are referred to as perfluorocarbons, which can be used in the present invention.

Examples of a vegetable oil include, but are not limited to, corn oil, coconut oil, cottonseed oil, olive oil, palm oil, peanut oil, rapeseed oil, safflower oil, sesame oil, soybean oil, sunflower oil, nut oils (e.g., almond oil, cashew nut oil, hazelnut oil, macadamia nut oil, walnut oil), etc.

In the context of the present invention, the term “mineral oil” refers to an open- chain saturated hydrocarbon compound represented by the general formula CₙH₂ₙ₊₂ (wherein n represents an integer of 1 to 20). Above all, preferred is hexadecane (n=16) and more preferred is liquid paraffin (n=16 to 20). The mineral oils used here including liquid paraffin are advantageous in that they are highly permeable to oxygen (gas). High permeability to oxygen gas allows oxygen supply to cells present in the oil, so that the viability of the cells in droplets is not impaired.

Although such a biocompatible oil or vegetable oil may be used alone, when the biocompatible oil or vegetable oil is mixed with a mineral oil, the mixing ratio of the biocompatible oil or vegetable oil to the mineral oil is 1:0.5 to 1:3, preferably 1:2. In a case where corn oil is used as a vegetable oil and liquid paraffin is used as a mineral oil, the mixing ratio is greater than 1 to less than 3, relative to corn oil which is set to 1. For example, the ratio of corn oil to liquid paraffin is preferably 1:2.

In the present invention, the biocompatible oil, vegetable oil and mineral oil may each be supplemented as appropriate with additives. For example, the vegetable oil (preferably corn oil) may be supplemented with lecithin, while the mineral oil may be supplemented with a surfactant, etc.

### (5) Preparation of Monodisperse Droplets

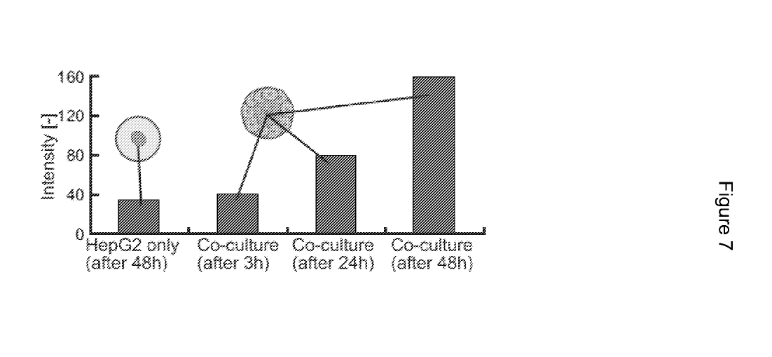

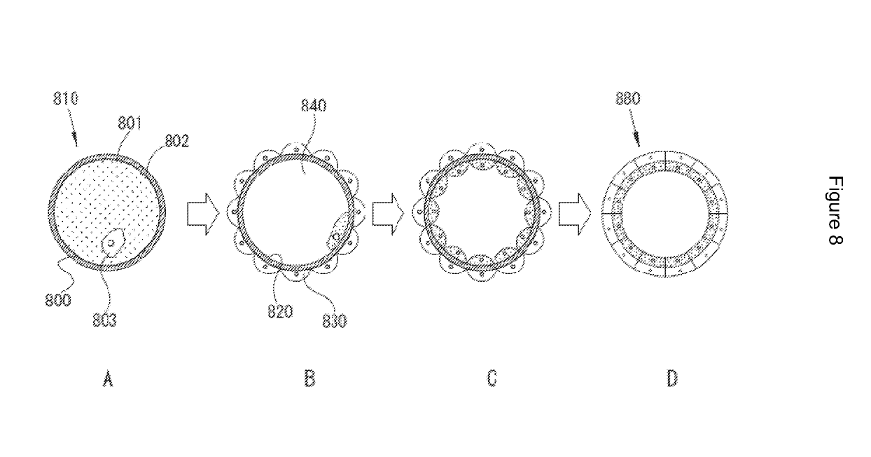

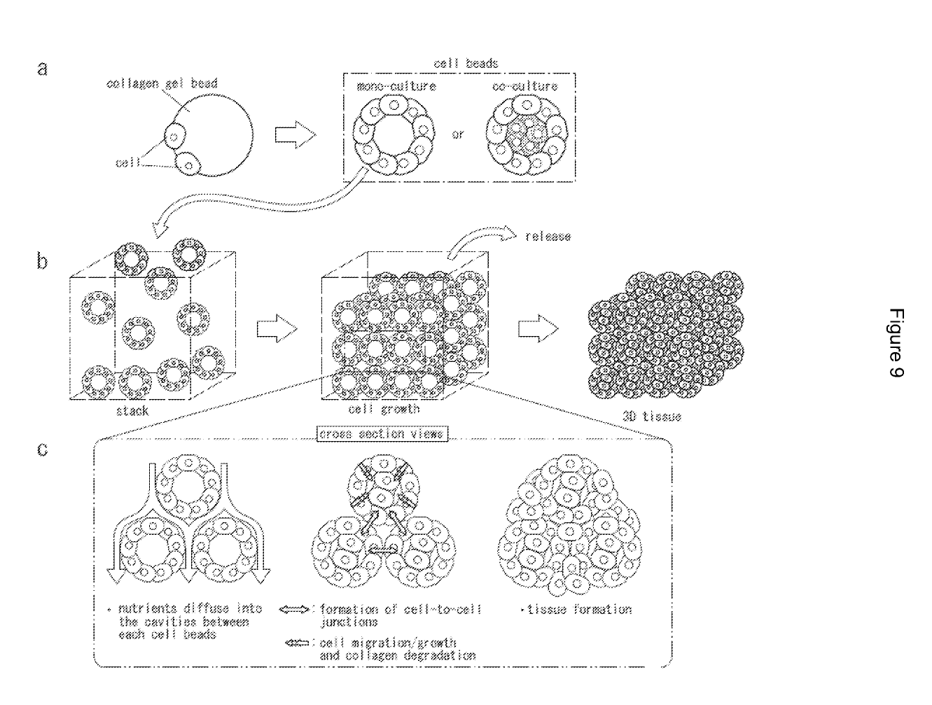

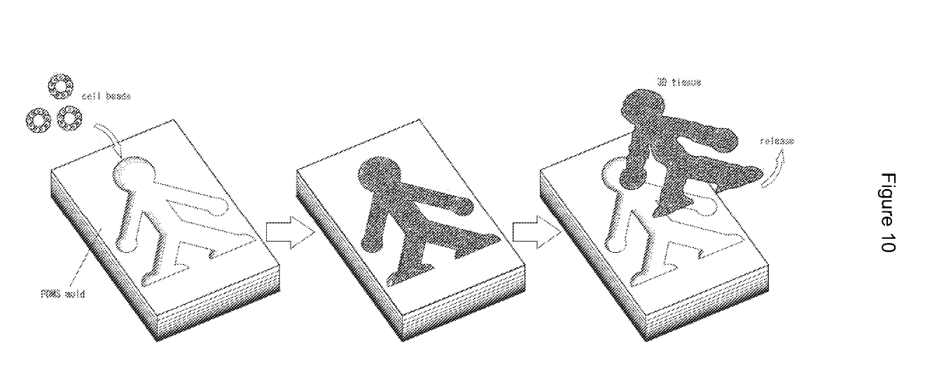

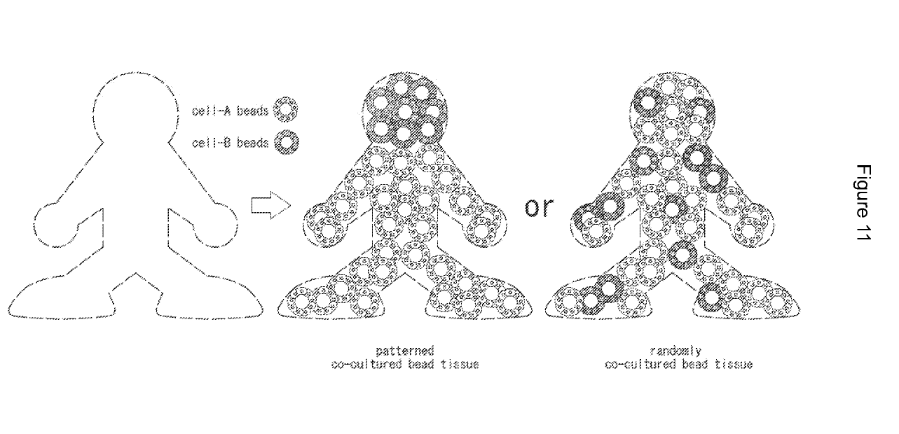

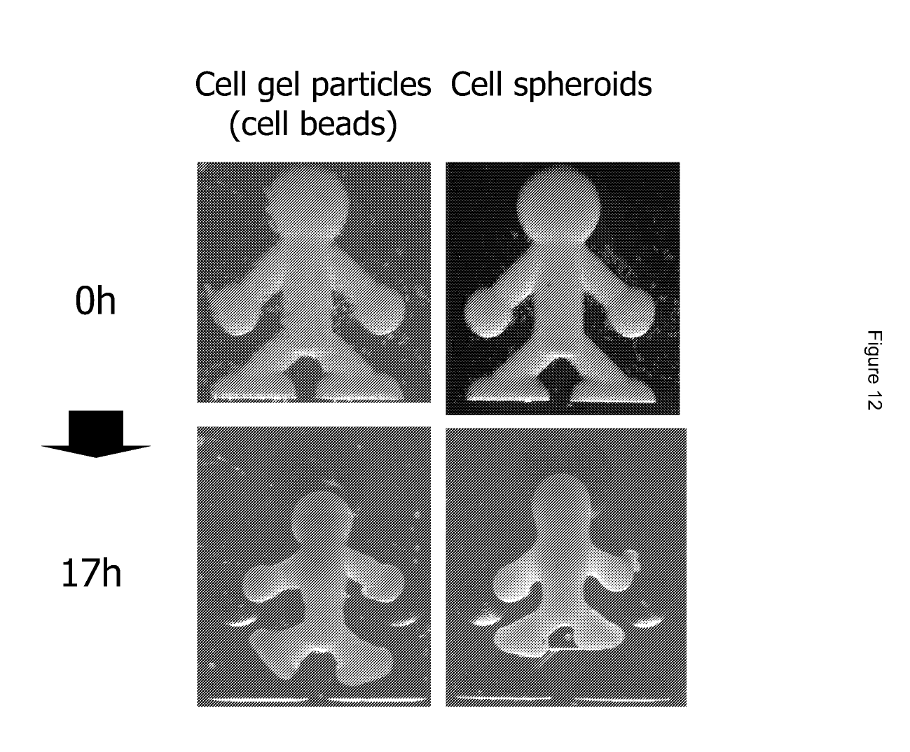

Any technique may be used for preparation of monodisperse droplets as long as a biocompatible oil or a vegetable oil or a mixture thereof with a mineral oil can be brought into contact with a solution containing a gel-forming component or a cell-containing solution. One of two microtubes is charged with a vegetable oil or an oil mixture and the other is charged with a cell-containing or cell-free solution, and the tubes are allowed to jet their contents under pressure, whereby these materials can be contacted with each other. In the present invention, a device called AFFD fabricated by stereolithography is preferred for use. The term “AFFD” refers to an axisymmetric flow-focusing device, which was developed by the inventors of the present invention (FIG. 2, Y. Morimoto et al., Proc. of MEMS 2008, pp. 304-307).

The use of AFFD has two advantages:

(i) the size of droplets can be altered by controlling the flow rate ratio of the outer fluid (continuous phase) to the inner fluid (dispersing phase); and

(ii) droplets can be formed regardless of solution components because the droplets do not contact with the surface of AFFD (S. Takeuchi et al., Adv. Mater., vol. 17, pp. 1067-1072, 2005; A. Luque et al., J. Microelectromech. Syst., vol. 16, pp. 1201-1208, 2007; A. S. Utada et al., Science, vol. 308, pp. 537-541, 2005).

FIG. 2 shows a schematic diagram of AFFD. In FIG. 2, an embodiment where cells are encapsulated within the core region is taken as an example for illustration.

In FIG. 2, AFFD is equipped with a first chamber 202 having a channel 201 for passing a biocompatible oil or a vegetable oil or a mixture thereof with a mineral oil (also referred to as the “oil or oil mixture”) and a second chamber 204 having a channel 203 for passing the above core cells 206 and a solution containing extracellular matrix components 207. The channel 201 in the first chamber 202 has a shape covering around the outside of the second chamber 204 over a given distance L from the outlet 205 of the second chamber 204 to the proximity of the channel 201, and the oil or oil mixture flowing through the channel 201 and the cell-containing solution flowing through the channel 203 meet each other at the outlet 205 of the second chamber to form monodisperse droplets 208 at the narrow orifice 209. In this case, the solution serves as a dispersing phase, while the oil or oil mixture serves as a continuous phase. The term “dispersing phase” is intended to mean a fluid whose flow is interrupted at the outlet 205 of the second chamber to yield droplets, and the term “continuous phase” is intended to mean a fluid whose flow is not interrupted and is continuously maintained in AFFD. The mechanism by which the cell-containing solution and the oil or oil mixture meet each other to form droplets is as follows. When the solution surrounded with the oil or oil mixture flows into the narrow orifice 209, the pressure applied to the solution will be increased. Once the solution has passed through the narrow orifice 209, the pressure will be removed at a time to give droplets. To prepare monodisperse droplets by allowing the oil or oil mixture and the cell-containing solution to meet each other, the number of capillaries at the outlet of the narrow orifice 209 should be set to 1 or less. Further, the flow rate of the oil or oil mixture in the present invention is set to 10 to 1500 μl/min, preferably 60 to 600 μl/min. On the other hand, the follow rate of the cell-containing solution is set to 1 to 20 μl/min, preferably 6 to 12 μl/min. When the ratio of these flow rates is 1 to 60, preferably 5 to 40, monodisperse droplets can be formed efficiently. The monodisperse droplets 208 thus formed are collected into a culture vessel (not shown) through the channel 210.

Subsequently, the formed monodisperse droplets may be heated or cooled or chemically treated to induce gelation of the droplets. Small beads obtained upon gelation are herein referred to as “gel beads.”

Conditions required for gelation will vary depending on the type of gel-forming component to be used, and hence are selected as appropriate for the type of gel-forming component. For example, in the case of using neutral collagen, gelation may be accomplished by warming at 37° C. for about 45 minutes. For gelation of alginic acid, calcium ions may be bound to the droplets, by way of example. For gelation of agarose, the temperature may be set as appropriate for the type of agarose, preferably set to about 25° C. or less, by way of example. In the case of gelatin, droplets may be kept at a temperature of about 15° C. or less, by way of example.

Moreover, conditions required for gelation are intended to vary the temperature in the case of using collagen, gelatin, Matrigel or the like as a gel-forming component, and intended to add calcium ions in the case of using a sodium alginate solution.

In the case of peptide gels, gelation may be accomplished when pH is varied or ions are added.

In the case of fibrin gels, gelation may be accomplished when an enzyme called thrombin is added.

In the case of synthetic polymer gels, gelation may be accomplished when a polymerization initiator is added or when a prepolymer solution is supplemented with a photocrosslinking agent and irradiated with ultraviolet or visible light.

After obtaining gel beads by gelation, cover cells are seeded on the surface of the gel beads. Cover cells may be seeded in any manner, for example, by mixing a cell suspension containing cover cells with the above gel beads obtained by gelation, or by adding such a cell suspension to a vessel containing the gel beads. Since cells have a tendency to adhere to the extracellular matrix, once the cover cells have been seeded, a biomaterial will be obtained in which the core cells are encapsulated within the gel beads and the cover cells are adhered to the surface layer of the gel beads. The biomaterial is then cultured to form a cell aggregate composed of two cell layers (FIG. 2). Culture conditions required to form two cell layers involve 24 to 48 hours, preferably 24 to 30 hours, at 37° C. in a 5% CO₂ atmosphere. The culture period may be set as appropriate for the size of the biomaterial and/or the amount of cells forming each layer.

### (6) Preparation of Hierarchical Biomaterials Having Multiple Types of Cell Layers

The preparation of hierarchical biomaterials having one or two cell layers has been described in (1) to (5) above. In this embodiment, third cells are seeded onto the biomaterial prepared above having hierarchically layered cells to form alternating cell layers.

Thus, the present invention provides a method for preparing a biomaterial comprising a gel layer which forms a core region or a gel layer which forms a core region where cells (core cells) are encapsulated, cells (cover cells) which cover around the gel layer, and an additional set of a gel layer and cover cells coated sequentially over the first-mentioned cover cells. The method of the present invention comprises the steps of:

(a) bringing a biocompatible oil or a vegetable oil or a mixture thereof with a mineral oil into contact with a solution containing a gel-forming component or with a solution containing the core cells and a gel-forming component to form monodisperse droplets;

(b) inducing gelation of the monodisperse droplets to give gel beads;

(c) seeding the cover cells over the surface of the gel beads; and

(d) preparing a mixture containing the biomaterial obtained in step (c) and a gel-forming component, and then repeating steps (a) to (c) by using the biomaterial as the core cells in step (a).

According to the method of the present invention, the biomaterial obtained above having two cell layers may be applied to the second chamber 204 in FIG. 2 for contact with an oil in the same manner as used to prepare the above monodisperse droplets 208, and then seeded with cells to thereby form a biomaterial composed of three cell layers. To prepare four or more cell layers, the above steps (a) to (c) may be repeated.

The cells (third cells) to be seeded outside the two cell layers may be of the same or different type from the above core cells or cover cells. In a case where a tissue (e.g., blood vessel, cornea) that functions through a complex network formed from multiple cell layers is artificially reproduced in vitro, the third cells are preferably of different type from the core cells and cover cells. For example, blood vessels have a triple-layer structure composed of, from the inside, the vascular endothelial layer, the smooth muscle layer, and the fibroblast layer. It is therefore possible to use vascular endothelial cells as core cells, smooth muscle cells as first cover cells, and fibroblasts as second cover cells (i.e., third cells). Likewise, corneas have a triple-layer structure composed of, from the outside, the corneal epithelial layer, the corneal parenchymal layer, and the corneal endothelial layer. It is therefore possible to use corneal endothelial cells as core cells, corneal parenchymal cells as first cover cells, and corneal epithelial cells as second cover cells.

On the other hand, in the case of tissues which require network formation like pancreatic islets to exert their functions, it is possible to use third cells of the same type as the first cells encapsulated within the gel. For example, pancreatic islets have a double-layer structure composed of, from the inside, a group of β cells and a group of α cells. Thus, β cells may be used as core cells, while α cells may be used as cover cells.

In a case where cells are not encapsulated within the core region, a biomaterial can also be prepared in the same manner as described above, except that gel beads are prepared without encapsulating any cells into the layer serving as a core region.

### (7) Production of Cell Aggregates

In the present invention, upon culturing the biomaterial obtained as described above, the cells can grow on the gel as a scaffold to obtain a cell aggregate composed of hierarchical cell layers in which the core cells are coated sequentially with multiple cell layers.

Culture procedures used to obtain cell aggregates may be standard cell culture procedures such as in-gel culture, shaking culture, in-microwell culture, hanging drop culture, etc. Culture conditions are as follows.

Culture period: 12 hours or longer, preferably 12 to 24 hours

Culture temperature: 37° C., by way of example

The cell aggregates thus obtained can be used as in vivo-like models of various tissues. For example, when using liver-derived cells or liver cancer cells as core cells and fibroblasts as cover cells, they provide an in vivo-like model of liver tissue. However, in vivo-like models are not limited to liver tissues and include the tissues illustrated in (6) above, as well as other tissues.

In some cases, the cells (cover cells) which cover around the gel layer may grow and migrate into the inside of the gel layer with the passage of culture time. By means of this event, the size (e.g., diameter) of the gel may be controlled in line with the cell's ability to grow or to degrade the gel, so that cell aggregates having a desired cell density can be obtained. For example, it is also possible to form cell aggregates having a single uniform cell density among individual cell aggregates.

### (8) Production of Reconstructed Tissues

In the present invention, the biomaterials or cell aggregates produced by the above method may be cultured within a mold of any shape, whereby the individual cell aggregates can be reconstructed to form a self-organized tissue. As used herein, the term “tissue” is also used to mean a tissue fragment which is a part of the tissue. In the present invention, tissue reconstruction may be accomplished through the state of cell aggregates or directly from the biomaterials.

FIG. 9 is a schematic representation showing a reconstructed tissue assembled from the cell aggregates of the present invention. In FIG. 9, panel a shows the concept of the process for producing a cell aggregate by culturing the biomaterial of the present invention, while panel b shows the concept of the process for producing a tissue reconstructed from cell aggregates. In FIG. 9b, when cell aggregates are cultured in a container of a given shape, the cells are grown and reconstructed to form a three-dimensional tissue. Since the cell aggregates retain their independence from one another, nutrient sources can be diffused and supplied into the cavities between the cell aggregates even when the cell aggregates are joined together tightly (FIG. 9c). This allows formation of cell-to-cell junctions and collagen degradation. As a result, the cell aggregates are assembled to form a tissue (FIG. 9c).

FIG. 10 is a process flow showing the method of the present invention. For example, a mold of a given shape is formed in a container made of a biocompatible material. FIG. 10 shows a mold whose shape resembles the human body. Into this mold, the cell aggregates of the present invention are introduced and cultured for a given period of time to reconstruct a tissue. Then, the tissue thus reconstructed is collected, thereby obtaining a reconstructed tissue having a three-dimensional shape (FIG. 10). Any material may be used as a mold as long as it is resistant to cell adhesion or has a surface modified to avoid cell adhesion. Examples include PDMS (polydimethylsiloxane, a kind of silicone resin), agarose gel, acrylamide gel and so on. The time required for reconstruction is not limited in any way. For example, in a case where cell aggregates are molded into a mold whose size is 7 mm long, 5 mm wide and 1.5 mm thick (deep) for production of a reconstructed tissue, the cell aggregates can be joined together within 1 hour after molding to form a reconstructed tissue. In the case of producing a tissue having a three-dimensional structure, the time required is 1 hour or longer, preferably 17 hours, in a 5% CO₂ environment at 37° C.

Cell aggregates used for tissue production may be of the same or different types. For example, when two different types of cell aggregates are used for tissue production, cell aggregate 1 may be placed in one site and cell aggregate 2 may be placed in another site for culture (middle panel in FIG. 11). For example, the middle panel in FIG. 11 represents a tissue in which the trunk is composed of cell aggregate A and the head is composed of cell aggregate B, while the right panel in FIG. 11 represents a tissue composed of a random mixture of cell aggregate A and cell aggregate B.

The tissues thus reconstructed are also provided as in vivo-like models in the present invention. For example, when using cell aggregates formed from liver-derived cells or liver cancer cells (as core cells) and fibroblasts (as cover cells), they provide an in vivo-like model of liver tissue. According to some embodiments of the present invention, it is also possible to obtain a reconstructed tissue having a uniform cell density throughout the tissue, e.g., when using cell aggregates having a desired cell density.

### 3. Preparation of Hollow Beads

In micro-level environments, to separate biomolecules from their external environment, the biomolecules are immobilized on a substrate by spotting or other procedures, or alternatively, the biomolecules are filled into holes microfabricated on a substrate and covered with a lid. However, such holes fabricated on a substrate cannot change their positions, which limits the range of possible experiments.

In the present invention, to prepare a mobile micromolecule while separating a biomolecule from its external environment, there is provided a method for preparing a hollow biomaterial comprising a gel layer which has a hollow cavity and forms a core region, a biomolecule or cells which are encapsulated within the hollow cavity, and cells (cover cells) which cover around the gel layer (FIG. 8). The method of the present invention comprises the steps of:

(a) bringing a biocompatible oil or a vegetable oil or a mixture thereof with a mineral oil into contact with a solution containing cells or a biomolecule and a gel-forming component to form monodisperse droplets;

(b) inducing gelation of the monodisperse droplets to give gel beads;

(c) coating the surface of the gel beads with another gel component different from said gel and then inducing gelation; and

(d) dissolving the gel obtained in step (b).

The above steps (a) and (b) are the same as described above in “2. Preparation of biomaterials,” except that not only cells, but also a biomolecule is to be encapsulated within the hollow cavity in step (a). In this embodiment, a biomaterial (e.g., microbead 800) formed by gelation of a first gel component 801 may further be coated with a second gel component 802, which is different from the above gel, to prepare a double-layered gel microbead 810 (FIG. 8A). In this stage, cells or a biomolecule 803 is encapsulated into the innermost of the microbead. This microbead is taken as an example to illustrate the biomaterial below.

Examples of the biomolecule intended in the present invention include proteins, enzymes, nucleic acids (e.g., DNA, RNA), peptides, antibodies, low-molecular-weight compounds, drugs and so on.

Examples of the second gel component 802 include agarose, alginic acid, PEG, PMMA and so on, which are conjugated with RGD peptide or fibronectin.

Then, the first gel 801 coated with the second gel component 802 is dissolved to form a hollow cavity 840 inside the second gel layer, whereby the cells or biomolecule is encapsulated within the hollow cavity. It should be noted that since the above “hollow cavity” refers to a space, the phrase “encapsulated within the hollow cavity” is intended to mean a state where the cells or biomolecule is in contact with the inner wall 820 of the gel layer (more specifically the inner wall surface of the second gel 802) (FIG. 8B). To dissolve the gel, a chelating agent may be used. For example, in the case of using a calcium alginate gel as a first gel component, once a chelating agent such as EDTA has been sprinkled over the gel, crosslinkages in the gel will be broken by the chelating effect between EDTA and calcium, and the broken gel will spontaneously leak outside the second gel component.

The microbead having a hollow cavity has the second gel 802 on its surface. Thus, when cells 830, which are different from the cells arranged within the hollow cavity, are seeded onto this bead and cultured in the same manner as described above, it is possible to prepare a microbead having a cell or biomolecule layer inside the gel and another cell layer outside the gel (FIG. 8C). It should be noted that the cells covering the outside of the second gel 802 may be seeded either before or after the first gel 801 is dissolved.

Then, the gel may be cultured to allow the cells to grow on the gel as a scaffold, thereby obtaining a cell aggregate 880 having two cell layers (FIG. 8D). Culture conditions required to obtain the cell aggregate 880 involve 24 to 48 hours at 37° C. in a 5% CO₂ atmosphere.

According to the method of the present invention, the gel membrane (the inner gel layer of the double-layered gel) may be removed depending on the object of experiments to form a hollow cavity inside the gel membrane. When cells or a biomolecule is arranged within the hollow cavity, the biomolecule can be separated from the external environment by being confined by the gel membrane. This extends the applicable range of experiments, etc.

The present invention will be described in more detail below by way of the following illustrative examples, which are not intended to limit the scope of the invention.

### EXAMPLE 1

### Preparation of the Biomaterials of the Present Invention

### Overview of Example 1

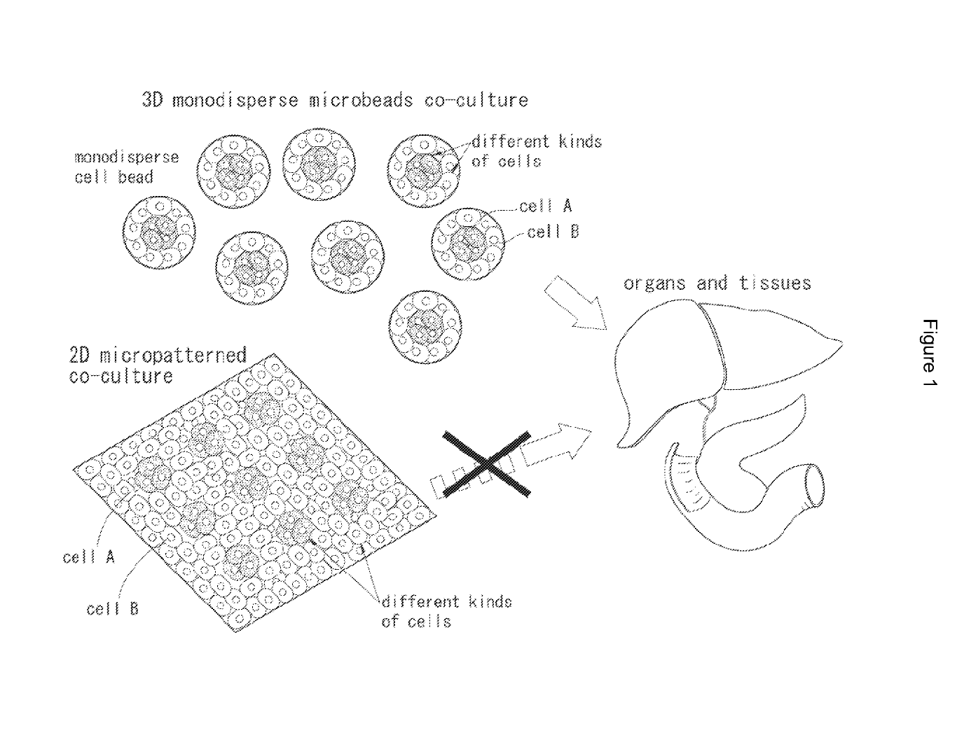

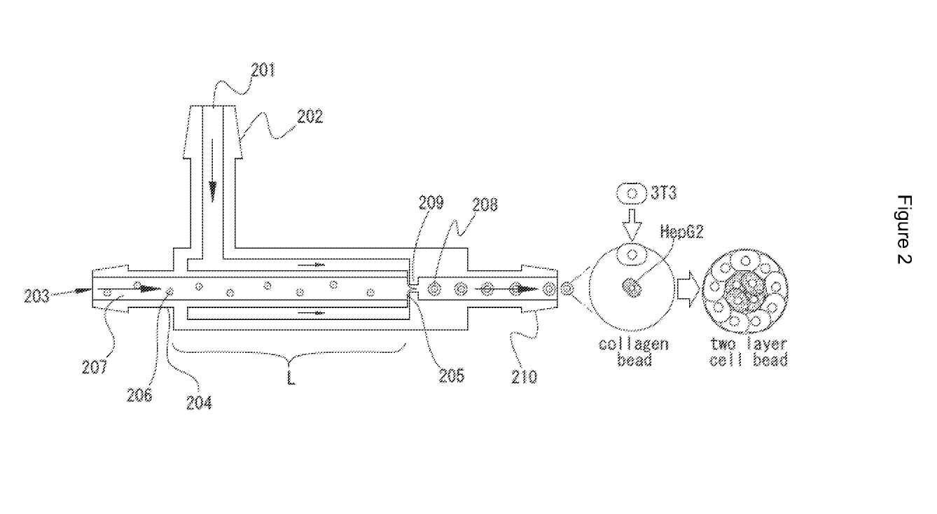

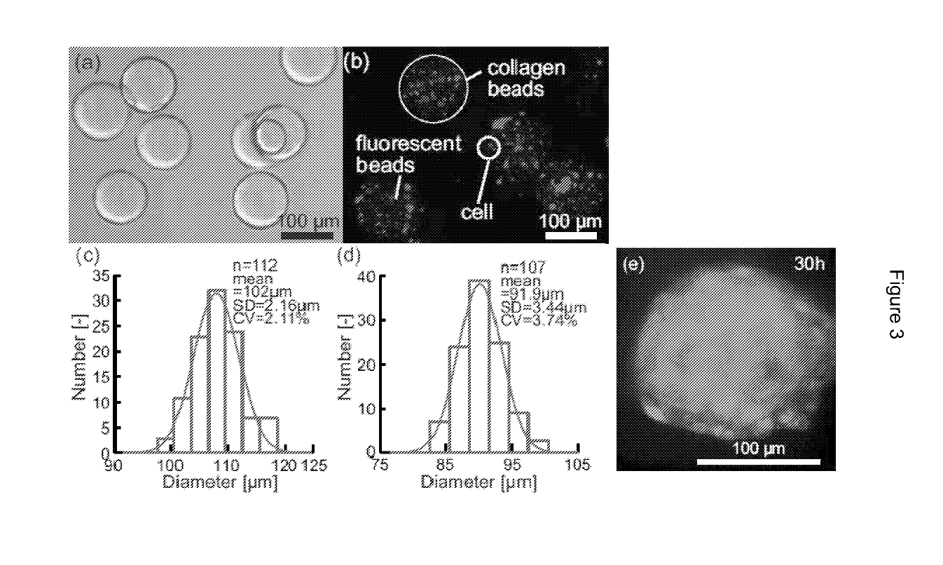

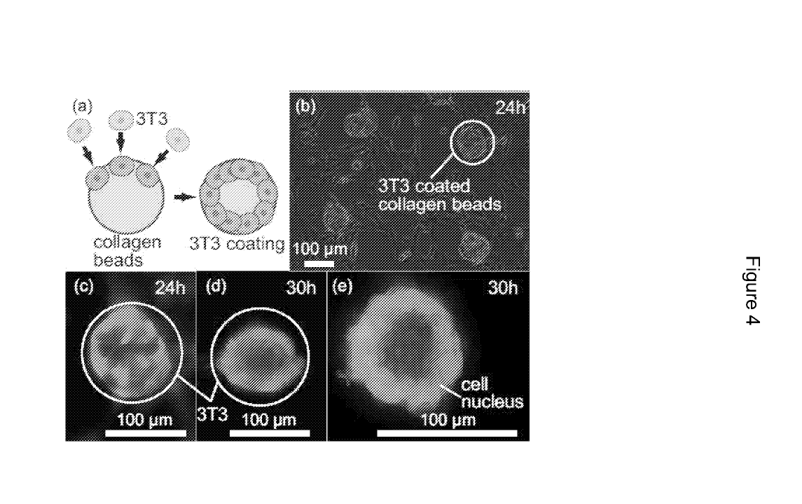

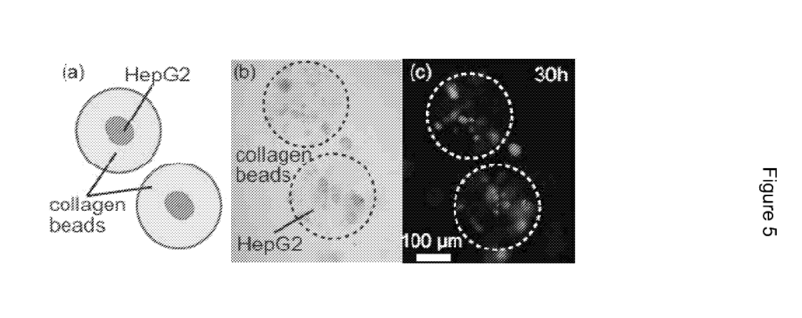

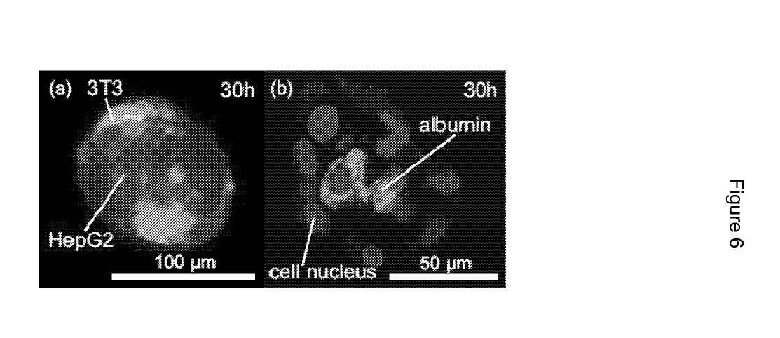

In this example, collagen, which is an extracellular matrix component, was used as a scaffold and different types of cells were arranged within and on collagen beads, respectively, in an attempt to reconstruct the native structure of each cell (FIG. 1, FIG. 2). Namely, collagen beads were used to construct a microtissue of HepG2 (hepatic cells) and 3T3 cells (fibroblasts) by being co-cultured three-dimensionally. When HepG2 cells were co-cultured with 3T3 cells, the HepG2 cells were able to secrete albumin at an increased level. This means that the system of the present invention has the ability to mimic tissue structures and functions in vitro.

### Materials and Methods

Corn oil (containing lecithin) and mineral oil were purchased from Wako Pure Chemical Industries, Ltd. Span80, Span20, Tween20 and hexadecane were purchased from Kanto Chemical Co., Inc. Dulbecco's modified eagle's medium (DMEM) and phosphate buffered saline (PBS) were purchased from SIGMA-Aldrich. A neutral collagen solution in DMEM (2 mg/ml) was purchased from KOKEN Co., Ltd. Other reagents were purchased from Kanto Chemical Co., Inc., Nacalai Tesque, Inc. and Wako Pure Chemical Industries, Ltd. Unless otherwise specified, water used in all experiments was obtained from a Millipore system having a specific resistance of 18 MΩ·cm.

### Cell Culture

The adherent cells used were 3T3 cells (mouse fibroblast-like cells) and HepG2 cells (human hepatoma cell line). The cells of each type were cultured under conditions of 37° C. and 5% CO₂ in DMEM which was supplemented with 10% (v/v) fetal bovine serum (Japan Bioserum) and 1% penicillin-streptomycin solution (SIGMA-Aldrich) for use as a culture medium. Fluorescent labeling of cells was performed with Cell Tracker Green CMFDA and Cell Tracker Orange CMTPX (Invitrogen), while cell nuclear staining was performed with Hoechst 33342 (Invitrogen). In addition, Live/Dead assay for determination of cell viability was performed with a LIVE/DEAD® Viability/Cytotoxicity kit (Invitrogen). These assays were each conducted according to the manufacturer's instructions.

### Preparation of Collagen Gel (as to Oil)

1. HepG2 cells were harvested from dishes and dispersed in a neutral collagen solution.

2. In AFFD, corn oil with lecithin (2 wt. %) was used as a continuous phase flow and the neutral collagen solution was used as a dispersing phase flow to prepare monodisperse droplets (cell-containing neutral collagen droplets of uniform diameter).

3. A 1:2 mixture containing corn oil with lecithin (2 wt. %) and liquid paraffin with Span 20 (1.5 wt. %) was introduced into microtubes, and the droplets were then injected into the mixture.

4. The droplets obtained in 3 were allowed to stand at 37° C. for 45 minutes to promote gelation of the collagen solution.

5. The gel was replaced in a cell culture solution according to procedures conventionally used to transfer gel beads from oil to water.

Formation of Collagen Sol Droplets with AFFD

AFFD fabricated by stereolithography was used to prepare monodisperse collagen sol droplets. The mechanism of monodisperse droplet formation and the fabrication process of AFFD are known (Y. Morimoto et al., Biomed. Microdev., DOI:10.1007/s10544-008-9243-y).

Briefly, the AFFD device has two concentric hollow cylinders. Each cylinder has a connection port that separately guides fluids such as oil and water into the device. These fluids are mutually immiscible and break up into droplets when the inner fluid (dispersing phase) streams through the outlet of the orifice (FIG. 2). The inner fluid (dispersing phase) is surrounded with the outer fluid (continuous phase) and the droplets formed are confined to the central axis of the microchannel. Since the flows of these dispersing and continuous phases do not contact with the channel surface, the problem of wetting on the inner wall surface can be avoided even when various droplets containing cells are produced. By varying the flow rates of the dispersing and continuous phases, the size of droplets can be adjusted.

The inventors of the present invention designed a device with three-dimensional modeling software (Rhinoceros, AppliCraft) and, to fabricate AFFD, used a commercially available stereolithography modeling machine (Perfactory, Envision Tec, Germany) with a photoreactive acrylate resin (“R11, 25-50 μm layer”) consisting of acrylic oligomer, dipentaerythritol pentaacrylate, propoxylated trimethylolpropane triacrylate, a photoinitiator and a stabilizer. It should be noted that the device was used in a vertical position.

Tefzel® tubes were attached to the inlet and outlet ports using silicon rubber tubes, and the dispersing and continuous phases were injected into the device through these tubes using syringe pumps (KDS-210, KD Scientific Inc., USA). The system was allowed to stabilize for one minute before collecting the droplets.

### Gelation of Collagen Droplets Encapsulating Cells

The monodisperse droplets prepared by AFFD are platforms for formation of monodisperse collagen gel beads encapsulating cells in a viable condition. In AFFD, corn oil with lecithin (2 wt %) was used for the continuous phase, while a neutral collagen solution in DMEM with cells was used for the dispersing phase. After monodisperse collagen sol droplets encapsulating cells were formed by AFFD, the droplets were collected into microtubes containing an oil mixture of corn oil with lecithin (2 wt %) and liquid paraffin with Span20 (2 wt %). Since a neutral collagen solution will be gelled under warmed conditions, the collagen solution was incubated in a water bath at 37° C. for 45 minutes to induce gelation of the collagen solution.

After collecting and warming the collagen gel beads in the oil mixture, the gel beads were transferred to DMEM in a known manner (W.-H. Tan and S. Takeuchi, Adv. Mater., vol. 82, pp. 364-366, 2003) to extract them from the oil. Namely, the collagen gel beads were deposited at the bottom of the microtubes, and the oil mixture around the gel beads was then removed by aspiration. Subsequently, hexadecane with Span80 (2 wt. %) was introduced into the microtubes to dissolve the remaining oil mixture for protecting the surface of the collagen gel beads from the oil sticking. Further, hexadecane was removed together with the oil mixture by aspiration, and DMEM with Tween20 (0.1 wt %) was added to separate the collagen gel beads from the oil. After the collagen gel beads were suspended in DMEM with Tween20 (0.1 wt%), the gel beads were collected by centrifugation. The supernatant was removed by aspiration and the gel beads were suspended again in DMEM. Centrifugation and suspension in DMEM were repeated for rinsing to obtain monodisperse collagen gel beads in DMEM.

### Results

### Monodisperse Collagen Gel Beads Containing Cells

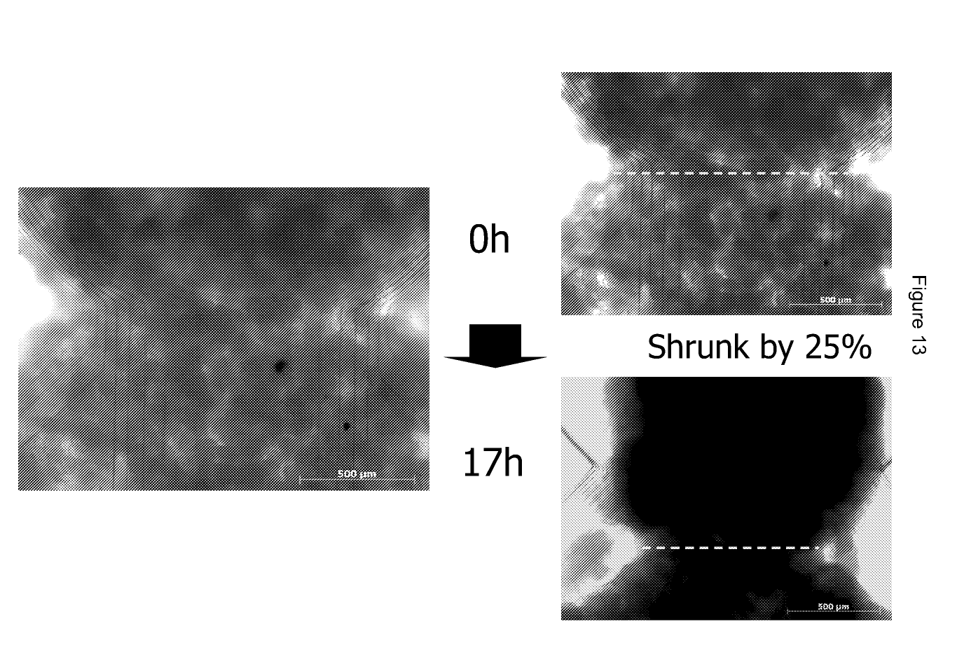

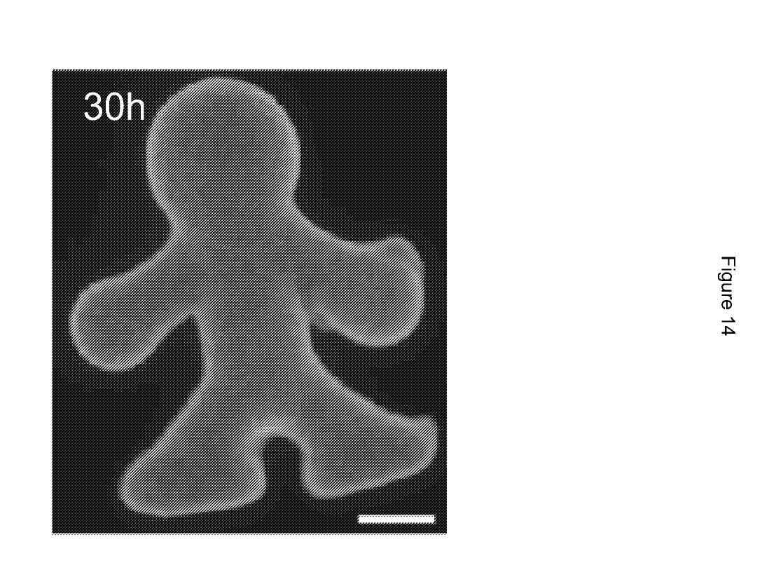

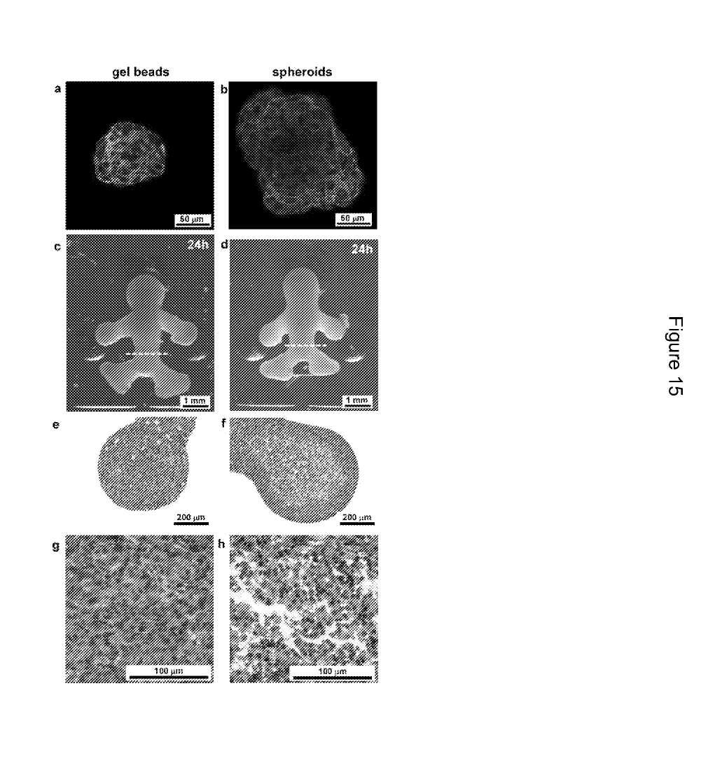

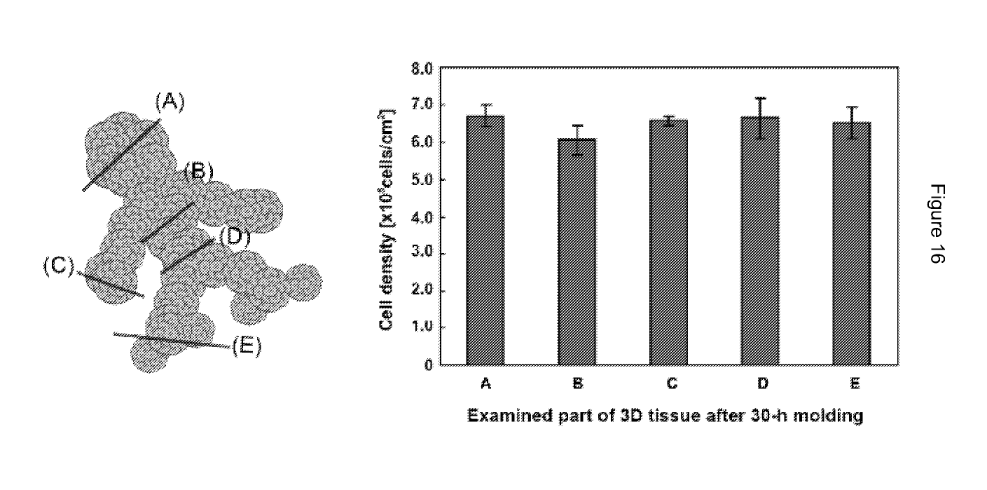

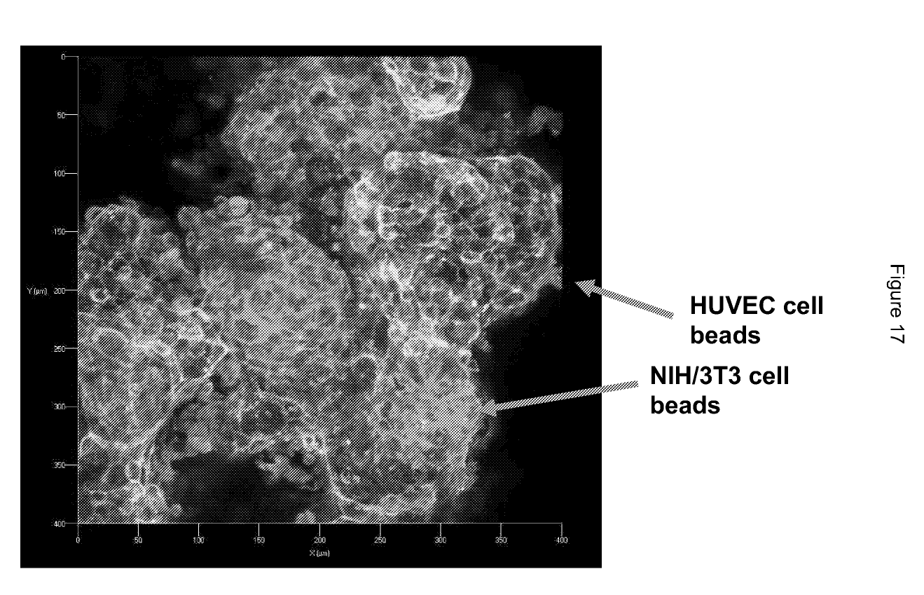

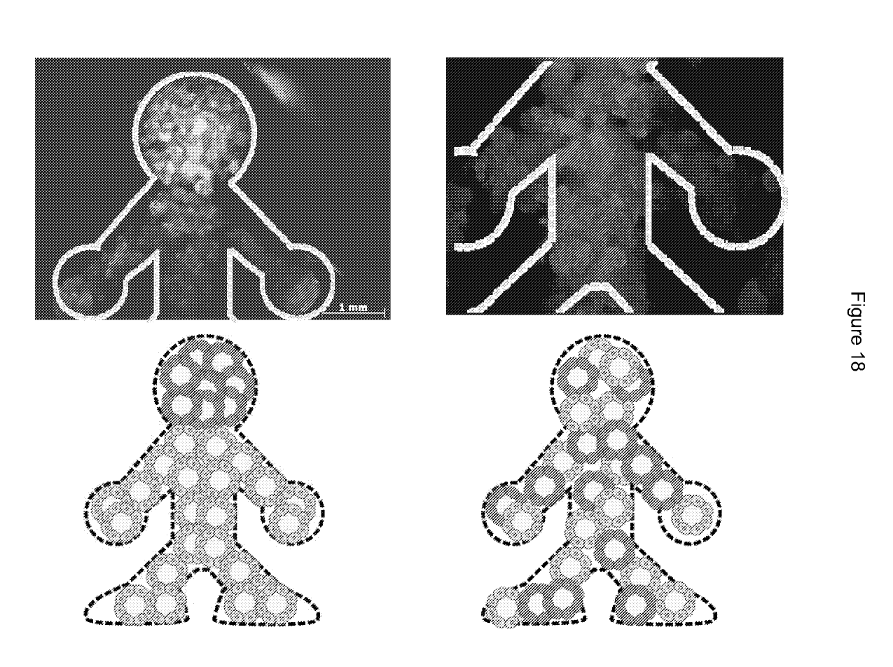

The inventors of the present invention prepared monodisperse collagen sol droplets and gel beads using AFFD fabricated by stereolithography. FIGS. 3(a) and 3(b) show monodisperse collagen sol droplets encapsulating cells, and collagen gel beads containing cells and fluorescent beads, respectively. FIG. 3(a) is an image showing collagen droplets during encapsulation of cells and collagen beads after encapsulation of cells, while FIG. 3(b) shows collagen gel beads after gelation.

In this example, the droplets and gel beads were prepared by setting the flow rates of the collagen solution and corn oil to 9 μl/minute and 60 μl/minute, respectively. FIGS. 3(c) and 3(d) show the diameter distribution of the collagen sol droplets and gel beads, respectively. It is indicated that the method of the present invention enables the formation of monodisperse collagen beads in which the encapsulated cells can retain their viability (e.g., FIG. 3(e)). Since the coefficient variation, which is defined as the ratio between the standard deviation and the mean, was less than 5%, it is recognized that both the collagen sol droplets and gel beads are monodisperse. Moreover, the encapsulated cells were found to retain their viability and were able to grow over 30 hours (FIG. 3(e)). The gelation method of the present invention is advantageous in that cells encapsulated within collagen gel can remain alive. If collagen sol droplets are surrounded with corn oil for 45 minutes, the monodispersity of the droplets will be maintained but cells in these droplets will die because corn oil does not have sufficient oxygen permeability. In contrast, if collagen sol droplets are surrounded with mineral oil for 45 minutes, cells will remain alive but the droplets of collagen will become polydisperse. Using an oil mixture of corn oil and mineral oil, the inventors of the present invention succeeded in gaining the advantages of these oils and hence were able to obtain collagen gel beads which maintain the viability of encapsulated cells while keeping the monodispersity of droplets.

### Mono-Culture Using Collagen Beads

### 3T3 Cells on the Surface of Collagen Gel Beads

The inventors of the present invention seeded 3T3 cells on the surface of monodisperse collagen gel beads. When the 3T3 cells were adhered onto the collagen gel beads (FIG. 4(a)), the cells grew and gradually covered the surface of collagen (FIGS. 4(b) to 4(d)). After the collagen gel beads were incubated for a given period of time, the cells were self-organized to form a 3T3 cell layer over the surface of the collagen gel beads. This result indicated that cell culture was possible on micro-sized spherical collagen.

FIG. 4(b) shows an image of monodisperse collagen gel beads coated with 3T3 cells. This image indicates that the monodispersity of collagen gel beads is maintained after adhesion of 3T3 cells. The 3T3 cells on the collagen gel beads gradually grew and migrated to form a layer of 3T3 cells (FIGS. 4(c) and 4(d)). The layer of 3T3 cells was formed after incubation for 30 hours, resulting in an outer layer composed of 3T3 cells (FIG. 4(e)).

HepG2 Cells within Collagen Gel Beads

In this example, HepG2 cells encapsulated within collagen gel beads prepared by the method of the present invention were cultured for 30 hours. As a result, most of the HepG2 cells within the collagen gel beads (cell aggregates) were alive, and the monodispersity of the collagen gel beads was maintained (FIGS. 5(a) to 5(c)). This result indicates that the method of the present invention does no harm to keep the activity of encapsulated cells (e.g., HepG2 cells).

Three-Dimensional Tissue Co-Culture of 3T3 cells and HepG2 Cells

Based on these results, when 3T3 cells were seeded on collagen beads (gel beads) encapsulating HepG2 cells and cultured for 30 hours, a cell aggregate consisting of HepG2 cells confined by a layer of 3T3 cells was formed. Namely, the inventors of the present invention succeeded in establishing a three-dimensional co-culture system for different types of cells (FIG. 6(a)). In this three-dimensional co-culture system, albumin secreted from HepG2 cells was visualized by immunostaining (A. Yamasaki et al., Hepatology, vol. 44, pp. 381A, 2006). As a result, the encapsulated HepG2 cells were found to secrete albumin (FIG. 6(b)), and their albumin secretion rate was increased due to the presence of 3T3 cells when compared to mono-culture of HepG2 cells (FIG. 7). Moreover, the level of albumin secretion from the HepG2 cells was increased in a manner dependent on the co-culture time with 3T3 cells, and was confirmed to be greater than that in the mono-culture system of HepG2 cells (FIG. 7). This cell aggregate constructed in vitro serves as a tissue model mimicking in vivo liver functions.

### Conclusion

In this example, 3T3 cells were seeded on the surface of collagen beads encapsulating HepG2 cells. As a result, it was observed that the HepG2 and 3T3 cells achieved single-size hierarchical cell co-culture to form a cell aggregate and cause intercellular interactions. In vitro three-dimensional co-culture beads are useful as in vivo-like tissue models for study of cell-cell interactions using various cells. Moreover, three-dimensional cell culture beads allow on-chip assays of chemicals/drugs.

According to the co-culture method of the present invention, it is possible to accurately control the spatial location of each cell and thus generate hierarchical tissue structures. In these beads, the inner cells are sufficiently confined by the outer cells. Three-dimensional co-culture of monodisperse collagen beads allows handling of these mobile tissues and provides a convenient experimental platform for biochemical/drug assays.

### EXAMPLE 2

### Production of Reconstructed Tissues

### Overview of Example 2

In this example, the biomaterial of the present invention (hereinafter also referred to as “cell gel beads”) was introduced into a mold in an attempt to form a reconstructed tissue. 3T3 cells were seeded as cover cells on the surface layer of collagen gel beads to form cell gel beads. When these cell gel beads were introduced into a millimeter-scale mold of any shape and then cultured, the individual beads were joined together via junctions formed between the cells on the surface layer of the gel beads to thereby reconstruct a three-dimensionally structured tissue of intended shape within 24 hours of culture. Tissue sections observed at 24 hours after reconstruction showed no necrosis within the tissue and a uniform cell density at each site. In contrast, when a reconstructed tissue was formed in the same manner from cell spheroids with high cell density, necrosis was observed within the tissue. Moreover, by varying the combination of cell gel beads, reconstruction of heterotissue structures was achieved.

In view of the foregoing, it was demonstrated that the method of the present invention was a simple and quick procedure allowing formation of a thick tissue with a uniform cell density. Moreover, since collagen gel beads are easy to change in size, the cell density in a tissue can be controlled freely and three-dimensional tissue construction can be customized to suit the proliferation or growth of each cell type. The contents of this example will be described in more detail below.

### Preparation of a Mold Used to Obtain Tissue Fragments

A convex human-shaped mold was prepared by stereolithography from an acrylic resin material. On the surface of this mold, a thin layer of paraxylylene resin was formed and a concave human-shaped mold was prepared from a silicone resin, polydimethylsiloxane (PDMS). The size of the mold was 7 mm long, 5 mm wide and 1.5 mm deep.

### Cell Culture

The adherent cells used were 3T3 cells (mouse fibroblasts) and human normal umbilical vein endothelial cells. The cells of each type were cultured under conditions of 37° C. and 5% CO₂ in DMEM which was supplemented with 10% (v/v) fetal bovine serum (Japan Bioserum) and 1% penicillin-streptomycin solution (SIGMA-Aldrich) for use as a culture medium.

### Production of Cell Aggregates

Collagen gel beads of about 100 μm diameter fabricated by AFFD were dispersed on non-cell-adhesive culture dishes, in which 3T3 or HUVEC cells were then seeded and cultured for 17 hours to prepare cell gel beads. In the case of 3T3 cells, they were cultured with shaking under conditions of 60 rpm. To produce cell spheroids, 3T3 cells were seeded on non-cell-adhesive culture dishes and cultured with shaking under conditions of 60 rpm.

### Molding of Cell Gel Beads

To produce a reconstructed tissue, the cell aggregates were poured into the mold prepared from PDMS (molding) and cultured under conditions of 37° C. and 5% CO₂, thereby obtaining a three-dimensionally reconstructed tissue. More specifically, after confirming that the cell aggregates were joined together at 1 or 2 hours after molding, an additional culture solution was further added and culture was continued in an incubator.

### Immunostaining and Visualization of Cells

The cell gel beads and cell spheroids were each fixed with 4% paraformaldehyde and treated to render the cell membrane permeable, followed by staining. For cytoskeleton, actin filaments were stained with Alexa488-labeled phalloidin (Invitrogen). Cell nuclei were stained with Hoechst 33342 (Invitrogen). In an experiment of heterotissue structure reconstruction with HUVEC cells and 3T3 cells, the HUVEC cells were reacted with anti-CD31 monoclonal antibody (Serotec) and stained with Alexa568-labeled anti-mouse IgG (Invitrogen) antibody.

### Preparation of Tissue Sections

After molding and culture for 24 hours, each reconstructed tissue was fixed with 4% paraformaldehyde. Tissue sections were prepared from the fixed tissue, and the prepared sections were stained with hematoxylin-eosin (H.E. staining). Based on H.E. stained images, the cell density and cell viability in the tissue were analyzed.

### Results

The results obtained are shown in FIGS. 12 to 18.

FIG. 12 shows tissues reconstructed from cell aggregates in a human-shaped mold. The left panel shows a tissue formed with the cell gel beads of the present invention, while the right panel shows a tissue formed with cell spheroids. FIG. 13 shows time-induced changes in a tissue obtained by the method of the present invention. At 17 hours after the initiation of reconstruction (culture), the tissue was found to shrink overall by about 25%. This is because the cell aggregates were joined together to give a dense state of cell aggregates per unit volume. Moreover, another possible factor is degradation of the collagen gel by the action of enzymes secreted from the cells per se.

FIG. 14 shows the results tested for the viability of cell aggregates in a tissue at 30 hours after the initiation of reconstruction (Live/Dead assay). Although green represents living cells and red represents dead cells, FIG. 14 shows no dead cell.

FIG. 15 shows tissue section images obtained for tissues reconstructed from cell gel beads and cell spheroids. FIGS. 15a and 15b show confocal images of cell aggregates in both cases, respectively. They are found to have different cell densities at the cell aggregate stage. Each was molded into a human-shaped mold to form a reconstructed tissue. FIGS. 15e to 15h show H.E. stained images of tissue sections in both cases. Purple represents cell nuclei. Red (pink) represents cytoplasm. Both tissues showed a uniform cell density at each site, but the tissue reconstructed from cell spheroids was found to lose the tissue interior and cell nuclei. This would be because the cell density is too high to supply each cell with oxygen and nutrients, thus leading to necrosis.

FIG. 16 shows the results of cell density at different sites determined from tissue sections prepared with cell gel beads. FIG. 16 indicated that the tissue had a uniform cell density when sectioned at any position.

FIG. 17 shows a three-dimensional heterotissue formed from HUVEC cell beads and NIH/3T3 cell beads by molding. After reconstruction, the HUVEC cell gel beads were stained red by immunostaining and their cytoskeleton was stained green. In addition, the cytoskeleton of the 3T3 cell gel beads was stained green. Namely, since the HUVEC cell gel beads were stained red and green, they are shown in red to yellow in the synthesized image of FIG. 17, while the 3T3 cell gel beads are shown in green only.

FIG. 18 shows tissues reconstructed by co-culture using two types of cell aggregates. A mixed color of red and green represents HUVEC cells, and green represents 3T3 cells. In the left panel, the head and trunk are found to be composed of different types of cells, respectively. In contrast, the right panel shows a tissue reconstructed by random introduction of HUVEC cells and 3T3 cells into a mold. In the right panel, red (yellow) (HUVEC cells) and green (3T3 cells) are found in admixture.

### INDUSTRIAL APPLICABILITY

The present invention provides biomaterials having hierarchical cell layers and a method for their preparation. Moreover, the method of the present invention enables the preparation of monodisperse gel droplets encapsulating cells. Cell aggregates and tissues obtained by the method of the present invention are useful as economical and convenient tools for in vitro study of in vivo-like microenvironments and cell-cell interactions.

1. A method for preparing a biomaterial comprising a gel layer which forms a core region, and cells (cover cells) which cover around the gel layer, said method comprising the steps of:
(a) bringing a biocompatible oil or a vegetable oil or a mixture thereof with a mineral oil into contact with a solution containing a gel-forming component to form monodisperse droplets;
(b) inducing gelation of the monodisperse droplets to give gel beads; and
(c) seeding the cover cells over the surface of the gel beads.

2. A method for preparing a biomaterial comprising a gel layer which forms a core region where cells (core cells) are encapsulated, and cells (cover cells) which cover around the gel layer, said method comprising the steps of:
(a) bringing a biocompatible oil or a vegetable oil or a mixture thereof with a mineral oil into contact with a solution containing the core cells and a gel-forming component to form monodisperse droplets;
(b) inducing gelation of the monodisperse droplets to give gel beads; and
(c) seeding the cover cells over the surface of the gel beads.

3. A method for preparing a biomaterial comprising a gel layer which forms a core region or a gel layer which forms a core region where cells (core cells) are encapsulated, cells (cover cells) which cover around the gel layer, and an additional set of a gel layer and cover cells coated sequentially over the first-mentioned cover cells, said method comprising the steps of:
(a) bringing a biocompatible oil or a vegetable oil or a mixture thereof with a mineral oil into contact with a solution containing a gel-forming component or with a solution containing the core cells and a gel-forming component to form monodisperse droplets;
(b) inducing gelation of the monodisperse droplets to give gel beads;
(c) seeding the cover cells over the surface of the gel beads; and
BIRCH, STEWART, KOLASCH & BIRCH, LLP GIVIM/GMM/sym
(d) preparing a mixture containing the biomaterial obtained in step (c) and a gel-forming component, and then repeating steps (a) to (c) by using the biomaterial as the core cells in step (a).

4. The method according to any one of claims 1 to 3, wherein the gel-forming component is an extracellular matrix component.

5. The method according to any one of claims 1 to 3, wherein the vegetable oil is corn oil.

6. The method according to any one of claims 1 to 3, wherein the mineral oil is liquid paraffin.

7. The method according to any one of claims 1 to 3, wherein the mixing ratio between the vegetable oil and the mineral oil is 1:2.

8. The method according to claim 4, wherein the extracellular matrix component is at least one member selected from the group consisting of collagen, proteoglycans, glycosaminoglycans, fibronectin, laminin, tenascin, entactin, elastin, fibrin, hyaluronic acid, gelatin, alginic acid, agarose and chitosan.

9. The method according to any one of claims 1 to 3, wherein the monodisperse droplets are formed when the oil mixture and the solution meet each other in a device comprising a first channel through which the oil mixture flows and a second channel through which the solution flows.

10. The method according to claim 2 or 3, wherein the core cells are derived from liver cancer and the cover cells are fibroblasts.

11. A method for producing a cell aggregate having hierarchically structured cell layers, which comprises culturing the biomaterial obtained by the method according to claim 1.

12. A method for producing a reconstructed tissue, which comprises culturing the biomaterial obtained by the method according to claim 1 or the cell aggregate obtained by the method according to claim 11 within a mold of any shape.

13. A method for preparing a hollow biomaterial comprising a gel layer which has a hollow cavity and forms a core region, a biomolecule or cells which are encapsulated within the hollow cavity, and cells (cover cells) which cover around the gel layer, said method comprising the steps of:
(a) bringing a biocompatible oil or a vegetable oil or a mixture thereof with a mineral oil into contact with a solution containing cells or a biomolecule and a gel-forming component to form monodisperse droplets;
(b) inducing gelation of the monodisperse droplets to give gel beads;
(c) coating the surface of the gel beads with another gel component different from said gel and then inducing gelation; and
(d) dissolving the gel obtained in step (b).

14. A method for producing a cell aggregate or biomolecular aggregate having a hollow cavity, which comprises culturing the hollow biomaterial obtained by the method according to claim 13.

15. A cell aggregate obtained by the method according to claim 11.

16. A tissue obtained by the method according to claim 12.

17. A cell aggregate or biomolecular aggregate having a hollow cavity obtained by the method according to claim 14.

18. A hierarchically structured cell aggregate comprising a gel layer which forms a core region, and a layer of cells which cover around the gel layer.

19. A hierarchically structured cell aggregate comprising a gel layer which forms a core region where cells (core cells) are encapsulated, and a layer of cells (cover cells) which are different from the core cells and cover around the core cell layer.

20. A hierarchically structured cell aggregate comprising a gel layer which forms a core region, a layer of cells (cover cells) which cover around the gel layer, and one or more cell layers coated sequentially with cells of the same or different type from the cover cells.

21. A hierarchically structured cell aggregate comprising a gel layer which forms a core region where cells (core cells) are encapsulated, a layer of cells (cover cells) which are different from the core cells and cover around the core cell layer, and one or more cell layers coated sequentially with cells of the same or different type from the cover cells.

22. The cell aggregate according to claim 19 or 21, wherein the core cells are HepG2 cells and the cover cells are 3T3 cells.

23. A reconstructed tissue assembled from the cell aggregates according to claim 18.

24. An in vivo-like model of liver tissue comprising the cell aggregate(s) according to claim 22 or a reconstructed tissue assembled from said cell aggregates.
